(19) United States
(12) Patent Application Publication
(10) Pub. No.: US 2026/0166312 A1
(43) Pub. Date: Jun. 18, 2026

(54) FLUORINE-FREE INSULATIVE BARRIER FOR IMPLANTABLE LEADS

(71) Applicant: Boston Scientific Scimed, Inc., Maple Grove, MN (US)

(72) Inventors: Andrew L. De Kock, Ham Lake, MN (US); Andrew J. Ro, Plymouth, MN (US); Justin Theodore Nelson, St. Paul, MN (US); Joseph Thomas Delaney, JR., Minneapolis, MN (US); Gene Thomas Storbeck, Millville, MA (US)

(21) Appl. No.: 19/416,707

(22) Filed: Dec. 11, 2025

Related U.S. Application Data
(60) Provisional application No. 63/733,935, filed on Dec. 13, 2024.

(51) Int. Cl. A61N 1/05

(52) U.S. Cl. CPC A61N 1/0573; A61N 1/0563

(57) ABSTRACT

An implantable lead for use with an implantable medical device (IMD), the implantable lead including a tubular lead body having a proximal end and a distal end, a proximal connector at the proximal end of the lead body configured for mechanically and electrically coupling the lead to the IMD, a helical electrode extending distally from a distal tip of a distal region of the lead body, and a conductor assembly positioned in a first lead body lumen in the tubular lead body. The conductor assembly includes an ultra-high-molecular-weight polyethylene (UHMWPE) material.

### CROSS REFERENCE TO RELATED APPLICATION

This application claims priority to U.S. Provisional Patent Application No. 63/733,935 entitled “FLUORINE-FREE INSULATIVE BARRIER FOR IMPLANTABLE LEADS,” filed Dec. 13, 2024, which is hereby incorporated by reference in its entirety.

### TECHNICAL FIELD

The present disclosure relates to medical electrical leads and associated manufacturing methods and methods of use. In particular, the present disclosure relates to implantable medical electrical leads for stimulating the conduction system of the heart.

### BACKGROUND

Various types of medical electrical leads for use in cardiac rhythm management (CRM) and neurostimulation systems are known. For CRM systems, such leads are typically extended intravascularly to an implantation location within or on a patient's heart, and thereafter coupled to a pulse generator or other implantable device for sensing cardiac electrical activity, delivering therapeutic stimuli, and the like. Long term reliability of such leads is important for the success of CRM, so the leads should be constructed to resist degradation, for example, from flexural fatigue resulting from patient movement. In some instances, the leads need to be removed from a patient after many years of service, and maintaining structural integrity of the lead is desirable so that the lead can be completely extracted in one piece.

### SUMMARY

In Example 1, an implantable lead for use with an implantable medical device (IMD), the implantable lead comprising: a tubular lead body having a proximal end and a distal end; a proximal connector at the proximal end of the lead body configured for mechanically and electrically coupling the lead to the IMD; a helical electrode extending distally from a distal tip of a distal region of the lead body; and a conductor assembly positioned in a first lead body lumen in the tubular lead body, wherein the conductor assembly comprises an ultra-high-molecular-weight polyethylene (UHMWPE) material.

In Example 2, the implantable lead of Example 1, wherein the conductor assembly comprises a conductor; and an insulator surrounding the conductor.

In Example 3, the implantable lead of Example 2, wherein the insulator comprises the UHMWPE material.

In Example 4, the implantable lead of Example 3, wherein the insulator consists essentially of the UHMWPE material.

In Example 5, the implantable lead of Example 4, wherein the insulator consists of the UHMWPE material.

In Example 6, the implantable lead of any of Examples 1-5, wherein the insulator does not contain fluoropolymer materials.

In Example 7, the implantable lead of any of Examples 2-6, wherein the conductor comprises a plurality of strands or filaments.

In Example 8, the implantable lead of Example 8, wherein each of the plurality of strands or filaments includes an individual insulator.

In Example 9, the implantable lead of Example 9, wherein each of the individual insulators comprises the UHMWPE material.

In Example 10, the implantable lead of any of Examples 1-9, wherein the first conductor assembly is electrically connected to one of the ring electrode and the helical electrode; and the implantable lead further comprises a second conductor assembly that is electrically connected to the other one of the ring electrode and the helical electrode.

In Example 11, the implantable lead of any of Examples 1-10, wherein the lead body comprises a polyurethane (PU) material or a silicone material.

In Example 12, the implantable lead of any of Examples 1-11, wherein the conductor assembly comprises a coiled conductor.

In Example 13, the implantable lead of Example 12, wherein the coiled conductor includes multiple filaments.

In Example 14, the implantable lead of any of Examples 1-11, wherein the conductor assembly comprises a straight single-stranded wire.

In Example 15, the implantable lead of any of Examples 1-11, wherein the conductor assembly comprises a stranded/braided cable conductor.

In Example 16, An implantable lead for use with an implantable medical device (IMD), the implantable lead comprising a tubular lead body having a proximal end, a distal end opposite the proximal end, and a first lead body lumen extending from the proximal end through the distal end; a proximal connector at the proximal end of the lead body configured for mechanically and electrically coupling the lead to the IMD; a distal region at the distal end of the lead body including a distal assembly, the distal assembly comprising: a ring electrode positioned around an exterior of a portion of the distal region; and a helical electrode extending distally from a distal tip of the distal region; a first conductor assembly positioned in a first lead body lumen in the tubular lead body, wherein the conductor assembly comprises an ultra-high-molecular-weight polyethylene (UHMWPE) material.

In Example 17, The implantable lead of Example 16, wherein the first conductor assembly comprises a conductor; and an insulator surrounding the conductor.

In Example 18, the implantable lead of Example 17, wherein the insulator comprises the UHMWPE material.

In Example 19, the implantable lead of Example 18, wherein the insulator consists essentially of the UHMWPE material.

In Example 20, the implantable lead of Example 19, wherein the insulator consists of the UHMWPE material.

In Example 21, the implantable lead of Example 16, wherein the insulator does not contain fluoropolymer materials.

In Example 22, the implantable lead of Example 17, wherein the conductor comprises a plurality of strands or filaments.

In Example 23, the implantable lead of Example 22, wherein each of the plurality of strands or filaments includes an individual insulator.

In Example 24, the implantable lead of Example 23, wherein each of the individual insulators comprises the UHMWPE material.

In Example 25, the implantable lead of Example 16, wherein the first conductor assembly is electrically connected to one of the ring electrode and the helical electrode; and the implantable lead further comprises a second conductor assembly that is electrically connected to the other one of the ring electrode and the helical electrode.

In Example 26, the implantable lead of claim 16, wherein the lead body comprises a polyurethane (PU) material or a silicone material.

In Example 27, an implantable lead for use with an implantable medical device (IMD), the implantable lead comprising a tubular lead body having proximal end, a distal end opposite the proximal end, and a first lead body lumen extending from the proximal end through the distal end; a proximal connector at the proximal end of the lead body configured for mechanically and electrically coupling the lead to the IMD; a distal region at the distal end of the lead body including a distal assembly, the distal assembly comprising: a ring electrode positioned around an exterior of a portion of the distal region; and a helical electrode extending distally from a distal tip of the distal region; a conductor assembly positioned in a first lead body lumen in the tubular lead body, wherein the conductor assembly comprises an ultra-high-molecular-weight polyethylene (UHMWPE) material.

In Example 28, the implantable lead of Example 27, wherein the conductor assembly comprises: a conductor; and an insulator surrounding the conductor.

In Example 29, the implantable lead of Example 28, wherein the insulator comprises the UHMWPE material.

In Example 30, the implantable lead of Example 29, wherein the insulator consists essentially of the UHMWPE material.

In Example 31, the implantable lead of Example 30, wherein the insulator consists of the UHMWPE material.

In Example 32, the implantable lead of Example 27, wherein the lead body comprises a polyurethane (PU) material or a silicone material.

In Example 33, a method of manufacturing an implantable lead for use with an implantable medical device (IMD), the method comprising forming a conductor assembly, wherein the conductor assembly comprises an ultra-high-molecular-weight polyethylene (UHMWPE) material; and stringing the conductor assembly into a tubular lead body of the implantable lead.

In Example 34, the method of Example 33, wherein forming the conductor assembly comprises coating a conductor with the UHMWPE material.

In Example 35, the method of Example 34, further comprising shaping the UHMWPE material after coating such that the UHMWPE material has a surface texture that is configured to reduce friction with a lumen of a body of the implantable lead.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram of a conduction system pacing (CSP) system, consistent with various aspects of the present disclosure.

FIG. 2 is a schematic partial cutaway side view of a lead of the CSP system of FIG. 1, consistent with various aspects of the present disclosure.

FIG. 3 is a perspective cross-sectional view of the lead as indicated by line 3-3 in FIG. 2, consistent with various aspects of the present disclosure.

FIG. 4 is a cross-sectional view of the lead as indicated by line 3-3 in FIG. 2, consistent with various aspects of the present disclosure.

FIGS. 5A and 5B are cross-sectional views of alternative conductor assemblies, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

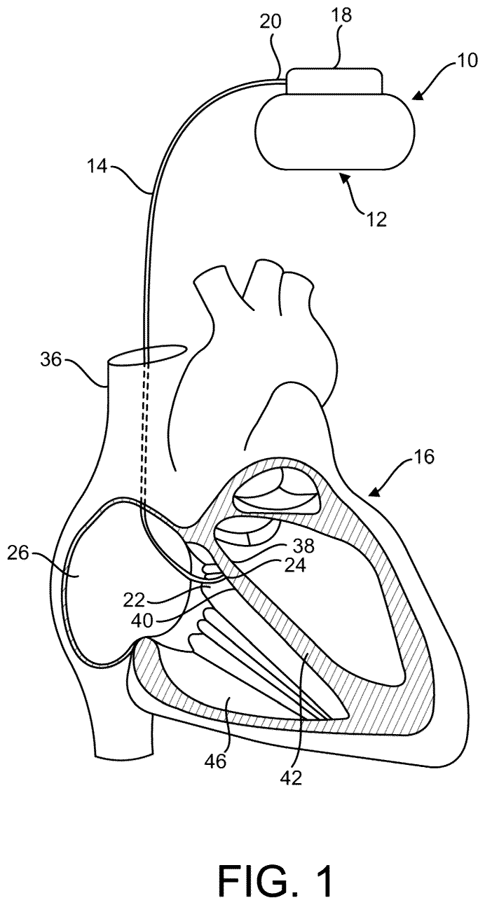

FIG. 1 illustrates a cardiac system pacing (CSP) system 10 including an implantable pulse generator 12 and a lead 14. The lead 14 is implanted in a heart 16. The implantable pulse generator 12 can include circuitry for sensing bioelectrical signals and/or delivering electrical stimulation via the lead 14. The implantable pulse generator 12 can include a lead interface 18 (e.g., a header). The lead 14 can include a proximal end 20, a distal end 22, and a fixation element 24 disposed at the distal end 22. In some embodiments, the pulse generator 12 is a pacemaker, a cardioverter/defibrillator, a cardiac resynchronization therapy (CRT) device, or a CRT device with defibrillation capabilities (a CRT-D device), among other appropriate devices. The pulse generator 12 is configured to be implanted subcutaneously within the body, for example, at a location such as in the patient's chest or abdomen, although other implantation locations are possible as well.

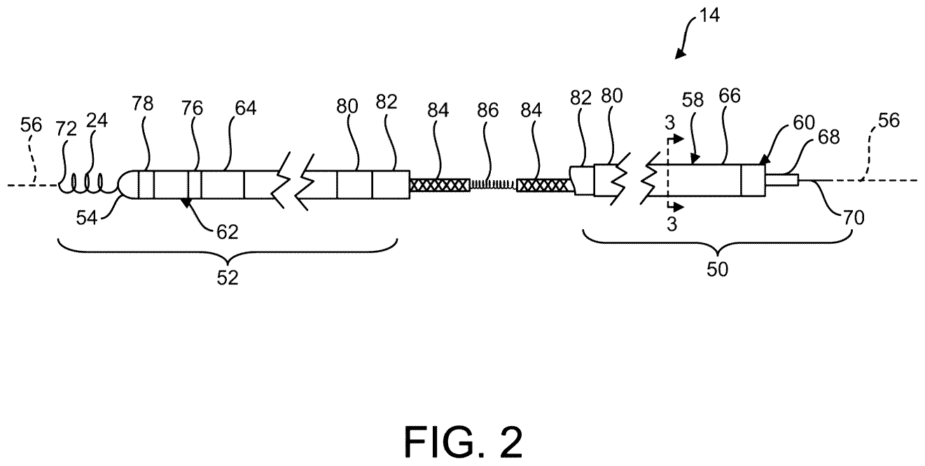

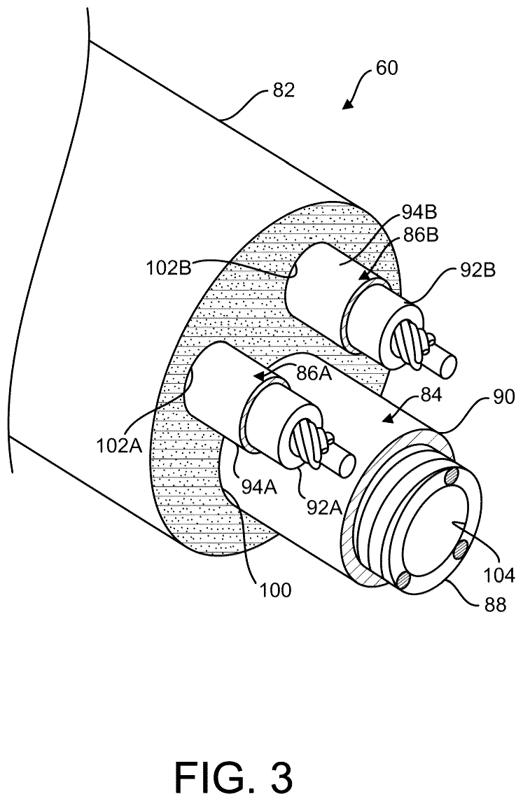

In the illustrated embodiment, the lead 14 further includes a proximal connector having one or more electrical contacts (shown in FIG. 2) at the proximal end 20, one or more electrical elements (e.g., ring electrodes) at the distal end 22 (shown in FIG. 2), and one or more electrical conductors (e.g., one or more coils or one or more cable conductors) (shown in FIG. 2) extending within one or more lumens (shown in FIG. 3) extending within the lead 14 from the electrical contacts to the electrical elements. The lead interface 18 can connect the pulse generator 12 to the electrical contacts at the proximal end 20 of the lead 14 to electrically connect the pulse generator 12 to the electrical elements. In other embodiments, the lead 14 is integrally formed with the pulse generator 12.

In the illustrated embodiment, the lead 14 is implanted in a right ventricle 46, at a ventricular septum 42, proximate a left bundle branch 38 and/or right bundle branch 40 of the specialized conduction system. The lead 14 operates to convey electrical signals between the target nerve(s), e.g., the left bundle branch 38 and/or the right bundle branch 40 and the implantable pulse generator 12. In some embodiments, the lead 14 enters the vascular system through a vascular entry site (not shown) formed in a wall of the left subclavian vein (not shown); extends through the left brachiocephalic vein (not shown), the superior vena cava 36, and the right atrium 26; and extends to the right ventricle 46. Other suitable vascular access sites are used in various other embodiments.

The fixation element 24 can fix the lead 14 to cardiac tissue, such as the area of tissue by which the left bundle branch 38 and/or the right bundle branch 40 can be directly stimulated. In some embodiments, the fixation element 24 can be electrically coupled to the implantable pulse generator 12 by, for example, one of the electrical conductors, such as a coil (shown in FIG. 2), extending to the proximal end 20 of the lead 14 for interfacing with the lead interface 18. As such, the fixation element 24 can mechanically and electrically couple the lead 14 to the tissue and facilitate the transmission of electrical energy from the conduction system in a sensing mode and to conduction system in a stimulation mode. In some embodiments, the fixation element 24 is a fixed fixation element, such as helix fixed to the lead 14. Such a fixation element 24 can be deployed by rotating the lead 14 itself to implant the fixation element 24 into the tissue. The use of the active fixation element for the fixation element 24 may allow for precise placement of the lead 14. The use of the active fixation element for the fixation element 24 may also provide for mapping capability because the user need not be concerned with accidental entanglement of the helix in the tissue.

While FIG. 1 only shows a single lead 14 connected to the implantable pulse generator 12 and implanted for cardiac stimulation, various other embodiments have one or more additional leads for sensing bioelectrical activity and/or stimulating other areas of the heart 16. For example, Boston Scientific corporation manufactures and markets several different types of implantable leads with the Reliance™ IS-1, Reliance™ DF4/IS-1/4, Reliance™ 4-FRONT, Fineline™ II, INGEVITY™, INGEVITY™+, ACUITY™ Spiral, ACUITY™ X4, and EMBLEM™ S-ICD electrodes that can each be connected to a lead of the present disclosure.

In some embodiments, the CSP system 10 can be capable of both pacing and defibrillation therapies. In such embodiments, the lead 14 can also include one or more high voltage defibrillation electrodes (shown in FIG. 2) for delivering defibrillation shocks capable of terminating ventricular fibrillation.

FIG. 2 is a schematic partial cutaway side view of the lead 14. In the illustrated embodiment, the lead 14 has a proximal region 50, a distal region 52 terminating at the fixation element 24 that extends from the distal tip 54, and a longitudinal axis 56. In general, the proximal region 50 is dimensioned so as to make up the portion of the lead 14 extending from the pulse generator 12 (shown in FIG. 1) to the location at which the lead 14 enters the right atrium 26 via the superior vena cava 36, whereas the distal region 52 is dimensioned to extend within the heart 16 (shown in FIG. 1) to the location at which the lead 14 is attached to the interior of the heart 16.

As shown, the lead 14 includes a flexible, elongate lead body 58, a proximal connector 60, and a distal assembly 62. The flexible elongate lead body 58 generally defines the longitudinal axis 56 of the lead 14. The lead body 58 has been partially dissected in FIG. 2 to show some of the internal components thereof. For example, the outer layer 80 forms the exterior of the lead body 58, and a core 82 is positioned just inside of the outer layer 80. The core 82 includes one or more lumens (shown in FIG. 3) and conductor assemblies 84, 86 are positioned inside the one or more lumens, respectively. The coil conductor assembly 84 includes a coil conductor 88 that is surrounded by a coil insulator 90, and in some embodiments, the coil conductor 88 is electrically connected to one or more of the conductors 70 and to the helical electrode 72 for transmitting electrical energy therebetween. The conductor assembly 86 includes a cable conductor 92 that is surrounded by a cable insulator 94, and in some embodiments, the cable conductor 92 is electrically connected to the one or more conductors 70 and to the shocking coil 74 and/or the ring electrode 76.

As further shown, the lead body 58 has a proximal end 64 and a distal end 66 opposite the proximal end 20. Additionally, the connector 60 is located at the proximal end 20 of the lead body 58, and the distal assembly 62 is located at and extends from the distal end 66 of the lead body 58. In some embodiments, the connector 60 includes a terminal pin 68 and one or more conductors 70 to electrically connect one or more active electrodes (e.g., the helical electrode 72 and/or the ring electrode 76, in some embodiments) to the implantable pulse generator 12. In some embodiments, the connector 60 is a conventional bi-polar connector. In other embodiments (e.g., having a quadripolar lead) the connector 60 and the one or more conductors 70 will be configured accordingly.

In the illustrated embodiment, the lead 14 includes a shocking coil 74 and a ring electrode 76 positioned around the exterior of a portion of the distal region 52. In some embodiments, the ring electrode 76 may be entirely surrounded by insulation rendering the ring electrode 76 inactive. The ring electrode 76 is mechanically and electrically connected to the implantable pulse generator 12 by an electrical conductor (à la conductor 70) that is joined to the ring electrode 76.

The distal region 52 also includes a helical electrode 72 that extends distally from the distal tip 54 of the lead 14. In embodiments, the helical electrode 72 is configured to operate as the fixation element 24 that can be rotated into tissue in order to fix the lead 14 to a desired portion of the interior of the heart 16. Additionally, in embodiments, the helical electrode 72 is configured to be used to sense the electrical activity of the heart 16 or to apply a stimulating pulse to the cardiac tissue. This would enable a physician to use the helical electrode 72 to map cardiac tissue and thereby identify an optimal attachment site. In other embodiments, the fixation element 24 is not electrically active and merely operates as a fixation means.

The distal region 52 also includes a drug collar 78 located on the distal assembly 62. The drug collar 78 includes an exposed surface and is impregnated with a drug or therapeutic. The drug collar 78 is configured to deliver a drug or therapeutic to a desired tissue within the heart 16. In some embodiments, the drug collar 78 is an overmolded collar, and in other embodiments, the drug collar 78 is a pre-formed collar.

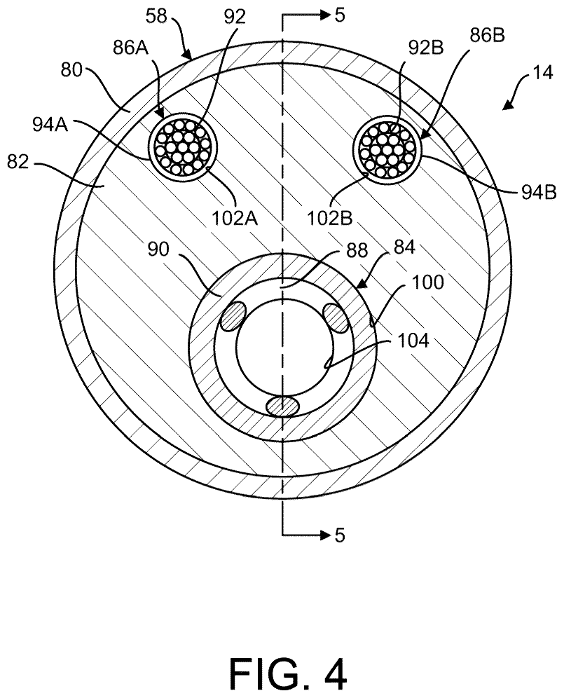

FIG. 3 is a perspective cross-sectioned view of the lead 14 as indicated by line 3-3 in FIG. 2. FIG. 4 is a cross-sectional view of the lead 14 as indicated by line 3-3 in FIG. 2. FIGS. 3 and 4 will now be discussed in conjunction with each other.

In the illustrated embodiment, the core 82 is a multi-lumen insulative member, which may be formed from a variety of non-conductive materials as are commonly used in implantable leads. As shown, the core 82 defines a coil lumen 100 in which the coil conductor assembly 84 is positioned. The core 82 also defines lumens 102 (i.e., 102A and 102B) in which the conductor assemblies 86 (i.e., 86A and 86B) are positioned, respectively. In some embodiments, the lumens 100, 102 have smooth, circular cross-sectional shapes, although in other embodiments, one or more of the lumens 100, 102 have different shapes and/or textures (including different shapes and/or textures from each other). The cable conductors 92 (i.e., 92A and 92B) of the conductor assemblies 86 are stranded/braided cables having a twelve-six-one configuration that are surrounded by the cable insulators 94 (i.e., 94A and 94B), although other configurations of the cable conductors 92 are possible (including straight, single-stranded wires).

The coil conductor 88 is a trifilar coil with a central lumen 104, although in other embodiments, the coil conductor 88 has a different number of filaments (e.g., unifilar, bifilar, quad-filar, and the like). In some embodiments, each of the filaments include their own individual layers of insulation, and in other embodiments, there is no insulation between the filaments. The coil conductor 88 is configured to conduct electrical signals, transmit torsional force (e.g., for rotating the lead 14 to implant the fixation element 24 into heart tissue), and transmit axial force (e.g., for repositioning or removal of the lead 14 from the patient). In addition, the central lumen 104 can accommodate other components, such as a stylet (not shown).

In the illustrated embodiment, the core 82 comprises a flexible, biocompatible material, such as, for example, polyurethane (PU) or silicone rubber. In some embodiments, the core 82 consists essentially of polyurethane (PU) or consists essentially of silicone rubber, and in some embodiments, the core 82 consists of polyurethane (PU) or consists of silicone rubber. In some embodiments, respective segments of the core 82 are made from different materials, so as to tailor the characteristics of the core 82 to its intended clinical and operating environments. The conductors 88, 92 comprise an electrically conductive material, for example, metal (e.g., nitinol, Elgiloy, MP35N, tungsten, tantalum, iridium, platinum, titanium, palladium, stainless steel, as well as alloys of these materials). In some embodiments, the conductors 88, 92 are low voltage and/or high voltage conductors. As used herein, “low voltage” conductors generally refer to conductors that are configured for low-voltage functions, such as sensing and pacing. “High voltage” conductors refer to conductors that are configured to conduct current at high voltages, as is required during defibrillation therapy, for example. In some embodiments, the coil conductor 88 is a low voltage conductor and is part of a system configured to provide pacing or CRT stimuli and/or to sense intrinsic cardiac electrical activity. In some embodiments, the cable conductors 92 are low voltage conductors and are used in the same system as the coil conductor 88 in the aforementioned embodiments. In some embodiments, the cable conductors 92 are high voltage conductors that are part of a system configured to provide antitachyarrhythmia therapy and/or cardioverter/defibrillator therapy.

In the illustrated embodiment, the conductors 88, 92 are surrounded and electrically isolated by insulators 90, 94. In some embodiments, the insulators 90, 94 have smooth, circular cross-sectional shapes, although in other embodiments, one or more of the insulators 90, 94 have different shapes and/or textures (including different shapes and/or textures from each other). In some embodiments, the insulators 90, 94 comprise an ultra-high-molecular-weight polyethylene (UHMWPE) material and do not contain any per-and polyfluoroalkyl substances (PFAS) (e.g., polytetrafluoroethylene (PTFE)). In some embodiments, each of the insulators 90, 94 are comprised of the same UHMWPE material, and in other embodiments, multiple different UHMWPE materials are used between the insulators 90 and/or 94 (e.g., UHMWPE materials that have different additives (e.g., colors) and/or are processed in different ways). In some embodiments, the insulators 90, 94 consist essentially of a UHMWPE material, and in some embodiments, the insulators 90, 94 consist of a UHMWPE material. UHMWPE materials have chain lengths that result in molecular weights in the scale of megadaltons (MDa or 10\hat{ }6 Da). In some embodiments, the ultra-long molecular lengths of UHMWPE materials have molecular weights in the range of about 0.50 MDa to about 25 MDa, in the range of about 1.0 MDa to about 12 MDa, or in the range of about 3.5 MDA to about 7.5 MDa. Such molecular lengths are in the scale of hundreds of thousands of monomer units long (e.g., between about 15,000 and 850,000 monomer units long, between about 30,000 and 400,000 monomer units long, or between about 100,000 and 250,000 monomer units long). In some embodiments, the thickness of the insulators 90, 94 is between about 0.0010 mm and about 0.20 mm, between about 0.0020 mm and about 0.15 mm, or between about 0.0025 mm and about 0.12 mm. In some embodiments, the dielectric strength of UHMWPE materials is between about 1.9 kV/mm to about 100 kV/mm or about 45 kV/mm, and in some embodiments, the insulators 90, 94 are configured to insulate conductors 88, 92 which have an electrical potential of between about 1.5 kV to about 2.6 kV.

In traditional leads, the conductors are electrically insulated using PFAS (e.g., PTFE). However, UHMWPE has advantages over these materials, for example, by being less environmentally problematic and having a higher wear resistance. UHMWPE materials also have differences and advantages over other forms of polyethylene, such as high-density polyethylene (HDPE) and cross-linked polyethylene (PEX). For example, the molecular weight of HDPE materials is in the scale of hundreds of thousands of daltons (Da) (e.g., about 100,000 Da to about 250,000 Da) with molecular lengths in the scale of thousands of monomer units long (e.g., about 700 to about 1,800 monomer units long). UHMWPE materials have higher lubricity, lower friction, and higher electrical insulation capability than HDPE or PEX materials. UHMWPE materials are more biologically stable and resistant to oxidation because the cross-linking sites in PEX materials are particularly susceptible to breakdown due to oxidation and free radical degredation. In addition, UHMWPE materials are less susceptible to fatigue issues than polyurethane (PU) materials. In some embodiments, the UHMWPE material on insulators 90, 94 is configured to survive at least 400,000,000 cycles of flexing of the lead 14.

However, UHMWPE also has some significant disadvantages compared to PTFE, HDPE, PEX, PU, and other polymers. For example, UHMWPE materials are inherently difficult to process. In particular, the molecules of UHMWPE materials are very long compared to other polymer materials, resulting in very high viscosity. Thus, it is not feasible to extrude such thin UHMWPE materials in a traditional manner despite traditional extrusion being an effective and widely used technique for processing other polymer materials. Instead, UHMWPE materials are processed using more aggressive, less reliable, and/or complicated methods, such as ram extrusion, compression molding, gel spinning or gel extrusion (e.g., extrusion using a sacrificial lubricant, such as, for example, decalin, xylene, or paraffin, to reduce the viscosity of the material, wherein the lubricant is removed later in the processing), either in a continuous reel-to-reel apparatus or as finite, short-length sections. However, none of these methods are appropriate for applying thin coatings. Furthermore, when coating a wire, braid, coil, or the like, any polymer coatings (e.g., insulation) already on the items can soften or melt due to the elevated temperatures at which UHMWPE material processing occurs.

In some embodiments, to make a thin film UHMWPE coating, the UHMWPE is essentially dissolved in a solvent at elevated temperatures. Then, the wire, braid, coil, or the like is dip coated in the solution (or another similar solvent casting process is performed).

Examples of suitable non-polar solvents include decalin, paraffin oil, p-xylene, pentane, hexane, heptane, limonene, dibutyl ketone, isophorone, dodecane, benzene, toluene, acetic acid, chloroform, diethyl ether, ethyl acetate, methylene chloride, dodecane, hexadecane, and pyridine, among others. In some embodiments, the non-polar solvent is selected from a group including decalin, paraffin oil, p-xylene, pentane, hexane, heptane, limonene, dibutyl ketone, isophorone, dodecane, benzene, toluene, acetic acid, chloroform, diethyl ether, ethyl acetate, methylene chloride, dodecane, hexadecane, and pyridine. In some embodiments, the non-polar solvent can be selected from a group including decalin, paraffin oil, p-xylene, along with any combination thereof. In some embodiments, the non-polar solvent can include decalin. In some embodiments, the non-polar solvent can include paraffin oil. In some embodiments, the non-polar solvent can include p-xylene.

In some embodiments, the non-polar solvent can consist essentially of decalin. In some embodiments, the non-polar solvent can consist essentially of paraffin oil. In some embodiments, the non-polar solvent can consist essentially of p-xylene. In some embodiments, the non-polar solvent can consist of decalin. In some embodiments, the non-polar solvent can consist of paraffin oil. In some embodiments, the non-polar solvent can consist of p-xylene.

In some embodiments, the lubricious polyethylene and the non-polar solvent can together form at least 70 weight percent, at least 80 weight percent, at least 90 weight percent, at least 95 weight percent, at least 99 weight percent of a total weight of the lubricious coating solution. For instance, in some embodiments, the lubricious polyethylene and the non-polar solvent can form an entirety (e.g., 100 weight percent) of the lubricious coating solution.

In some embodiments, a mixture of the non-polar solvent and the lubricious polyethylene polymer can be heated to an elevated temperature in a range from about 120 degrees Celsius to about 200 degrees Celsius. All individual ranges and sub-ranges are included. For instance, the mixture of the non-polar solvent and the lubricious polyethylene polymer can be heated to an elevated temperature in a range from about 130 degrees Celsius to about 150 degrees Celsius. In some embodiments the elevated temperature can be about 120 degree Celsius, about 125 degrees Celsius, about 130 degrees Celsius, about 135 degrees Celsius, 140 degrees Celsius, about 145 degrees Celsius, about 150 degrees Celsius, about 155 degrees Celsius, about 160 degrees Celsius, about 165 degrees Celsius, about 170 degrees Celsius, about 175 degrees Celsius, about 180 degrees Celsius, about 185 degrees Celsius, about 190 degrees Celsius, about 195 degrees Celsius, or about 200 degrees Celsius.

In some embodiments, the mixture of the non-polar solvent and the lubricious polyethylene polymer is agitated. For example, a magnetic stir-bar or other agitation mechanism is employed to agitate the mixture of the non-polar solvent and the lubricious polyethylene polymer.

Without wishing to be bound by theory, it is believed that heating the mixture of the non-polar solvent and the lubricious polyethylene polymer to an elevated temperature (e.g., an elevated temperature in a range from about 120 degrees Celsius to about 200 degrees Celsius, an elevated temperature in a range from about 120 degrees Celsius to about 160 degrees Celsius, or an elevated temperature in a range from about 130 degrees Celsius to about 150 degrees Celsius) promotes formation of a lubricious coating solution (e.g., it promotes dissolving of the lubricious polyethylene polymer in the non-polar solvent), particularly while the mixture is also agitated with an agitation mechanism. In some embodiments the non-polar solvent is heated to an elevated temperature and the lubricious polyethylene polymer (e.g., UHMWPE) is subsequently added to the heated non-polar solvent. The resultant mixture is agitated and continues to be heated substantially at the elevated temperature until the lubricious polyethylene polymer is dissolved in the non-polar solvent and the lubricious coating solution is formed.

Without wishing to be bound by theory, the presence of the particular non-polar solvents described herein improve processability (e.g., polymer solution rheology) and/or deliverability of the lubricious polyethylene polymer (e.g., UHMWPE) to the substrate. In some embodiments, the resultant lubricious coating solution has a relatively uniform dispersion of the lubricious polyethylene polymer in the non-polar solvent, which promotes, for example, formation of a relatively thin (e.g., about 1 micrometer to about 50 micrometers) yet durable and lubricious coating on a substrate (e.g., wire, braid, coil, or the like) of a medical device. In some such embodiments, the lubricious coating solution is employed to solution cast or otherwise form a thin lubricious coating on the substrate of the medical device.

While FIGS. 3 and 4 show the core 82 having three lumens 100, 102 and three elongate components (i.e., the conductors 84, 86), different numbers of lumens and elongate components are possible. For example, in some embodiments where the coil conductor assembly 84 is not needed to conduct electricity, the coil conductor 88 can be eliminated. In such embodiments, the coil insulator 90 can fulfil the roles of transmitting torsional and/or axial forces through the lead body 58. In such embodiments, the size (and therefore, strength) of the coil insulator 90 can be adjusted accordingly. In addition, in some embodiments, some or all of the cable conductors 92 92 have a coiled configuration instead of a stranded/braided configuration.

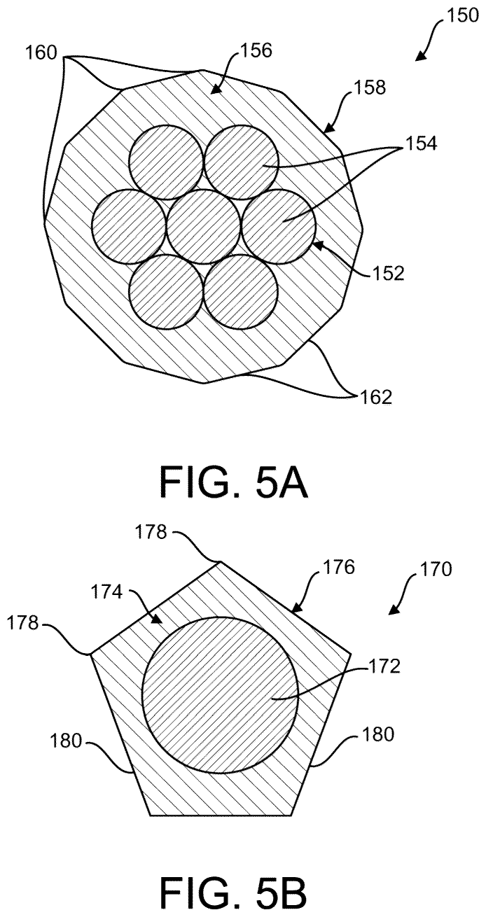

FIG. 5A shows a cross-sectional view of alternative conductor assembly 150. The conductor assembly 150 can represent (with or without modifications) any of the conductor assemblies 84, 86 (shown in FIG. 4). In the illustrated embodiment, the conductor assembly 150 has a cable conductor 152 comprising a plurality of strands 154 (e.g., in a six-one configuration), although other configurations of the cable conductor 152 are possible (including single-stranded wires). The cable conductor 152 is surrounded and covered by a cable insulator 156. In other embodiments, instead of or in addition to the cable insulator 156, each strand 154 includes its own individual insulation layer comprising, consisting essentially of, or consisting of UHMWPE material. The UHMWPE material used for each individual insulation layer can be the same as or different from the UHMWPE material used in the cable insulator 156.

In the illustrated embodiment, the cable insulator 156 has an exterior surface 158 with a texture such that the exterior surface 158 has a dodecagonal shape. In some embodiments, the texture is in the form of a series of twelve raised elongate edges 160 at the intersections of adjacent ones of the twelve flat sides 162, although other configurations of the exterior surface 158 are possible (e.g., greater or fewer numbers of edges 160 and sides 162). The edges 160 are roughly parallel to or wind helically around the longitudinal axis of the cable conductor 152. In other embodiments, other types of surface texture are used on the exterior surface 158. In some such embodiments, the exterior surface 158 includes distinct, discontinuous, raised features such as, for example, micron-scale ridges or micronodules of any appropriate tip shape (e.g., rounded, flat-topped, or angular). In some such embodiments, the surface texture is non-uniform. In some embodiments, the surface texture is directionality aligned with either or both the longitudinal axis of the cable conductor 152 or the axis perpendicular thereto.

In some embodiments, adding texture or roughness to the exterior surface 158 results in different surface characteristics compared to the smooth exterior surfaces of the insulators 90, 94 (shown in FIG. 4). In particular, having a textured exterior surface 158 abutting smooth interior surfaces of lumens 100, 102 (shown in FIG. 4) has been found to minimize frictional forces between these respective surfaces when they contact one another. The presence of the texture on the external surface 158 of the conductor assembly 150, and the corresponding reduction in frictional resistance with respect to the adjacent inner surface of the lumens 100, 102, can also increase the manufacturability and ease of assembly of the lead 14 (shown in FIG. 4). During the manufacture of the lead 14, certain processing aids (e.g., vacuum, alcohol or other solvents, and/or pressurized gases) are generally used in order to string one component co-radially through another, such as the conductor assembly 150 through the core 82 (shown in FIG. 4). The use of such processing aids can be time-consuming, costly, and/or ineffective at reducing friction. Therefore, use of such processing aids by reducing the friction between components during assembly of the medical devices or systems is beneficial.

In some embodiments, the specific configuration of the surface texture on the external surface 158 is based upon factors such as, without limitation, the type and/or size of the conductor assembly 158 and the lumens 100, 102; the proximity of (e.g., clearance between) the external surface 158 and the internal surface of the lumens 100, 102; and other design and manufacturing considerations applicable to the lead 14. In some embodiments, a substantially smooth inner surface of the lumens 100, 102 has a roughness average (Ra) of less than 10 microinches, and the average surface texture roughness on the external surface 158 is greater than about 16 microinches.

In some embodiments, the external surface 158 can initially have a smooth surface texture which is altered by a manufacturing process to form a different texture. Such a manufacturing process can be, for example, an embossing, grit-blasting (e.g., using dry ice particles), die drawing while the cable insulator 156 is malleable (e.g., still warm, prior to solidification).

In other embodiments, the internal surfaces of some or all of the lumens 100, 102 include texture such as that described with respect to the external surface 158. Such embodiments can be paired with a textured external surface 158 or with a smooth external surface 158 (à la the insulators 90, 94).

FIG. 5B shows a cross-sectional view of alternative conductor assembly 170. The conductor assembly 170 can represent (with or without modifications) any of the conductor assemblies 84, 86 (shown in FIG. 4). In the illustrated embodiment, the conductor assembly 170 has a single-wire conductor 172, and the conductor 172 is surrounded and covered by an insulator 174.

In the illustrated embodiment, the insulator 174 has an exterior surface 176 with a texture such that the exterior surface 176 has a pentagonal shape. In some embodiments, the texture is in the form of a series of five raised elongate edges 178 at the intersections of adjacent ones of the five flat sides 180, although other configurations of the exterior surface 176 are possible (e.g., greater or fewer numbers of edges 178 and sides 180). The edges 178 are roughly parallel to or wind helically around the longitudinal axis of the conductor 172. In other embodiments, other types of surface texture are used on the exterior surface 176. In some such embodiments, the exterior surface 176 includes distinct, discontinuous, raised features such as, for example, micron-scale ridges or micronodules of any appropriate tip shape (e.g., rounded, flat-topped, or angular). In some such embodiments, the surface texture is non-uniform. In some embodiments, the surface texture is directionality aligned with either or both the longitudinal axis of the conductor 172 or the axis perpendicular thereto. In some embodiments, the surface texture of the exterior surface 176 functions similarly to or the same as that of the conductor assembly 150 (shown in FIG. 5A), and in some embodiments, the surface texture of the exterior surface 176 can be present in some or all of the lumens 100, 102.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An implantable lead for use with an implantable medical device (IMD), the implantable lead comprising:
a tubular lead body having a proximal end, a distal end opposite the proximal end, and a first lead body lumen extending from the proximal end through the distal end;
a proximal connector at the proximal end of the lead body configured for mechanically and electrically coupling the lead to the IMD;
a distal region at the distal end of the lead body including a distal assembly, the distal assembly comprising:
a ring electrode positioned around an exterior of a portion of the distal region; and
a helical electrode extending distally from a distal tip of the distal region;
a first conductor assembly positioned in a first lead body lumen in the tubular lead body, wherein the conductor assembly comprises an ultra-high-molecular-weight polyethylene (UHMWPE) material.

2. The implantable lead of claim 1, wherein the first conductor assembly comprises:
a conductor; and
an insulator surrounding the conductor.

3. The implantable lead of claim 2, wherein the insulator comprises the UHMWPE material.

4. The implantable lead of claim 3, wherein the insulator consists essentially of the UHMWPE material.

5. The implantable lead of claim 4, wherein the insulator consists of the UHMWPE material.

6. The implantable lead of claim 1, wherein the insulator does not contain fluoropolymer materials.

7. The implantable lead of claim 2, wherein the conductor comprises a plurality of strands or filaments.

8. The implantable lead of claim 7, wherein each of the plurality of strands or filaments includes an individual insulator.

9. The implantable lead of claim 8, wherein each of the individual insulators comprises the UHMWPE material.

10. The implantable lead of claim 1, wherein:
the first conductor assembly is electrically connected to one of the ring electrode and the helical electrode; and
the implantable lead further comprises a second conductor assembly that is electrically connected to the other one of the ring electrode and the helical electrode.

11. The implantable lead of claim 1, wherein the lead body comprises a polyurethane (PU) material or a silicone material.

12. An implantable lead for use with an implantable medical device (IMD), the implantable lead comprising:
a tubular lead body having proximal end, a distal end opposite the proximal end, and a first lead body lumen extending from the proximal end through the distal end;
a proximal connector at the proximal end of the lead body configured for mechanically and electrically coupling the lead to the IMD;
a distal region at the distal end of the lead body including a distal assembly, the distal assembly comprising:
a ring electrode positioned around an exterior of a portion of the distal region; and
a helical electrode extending distally from a distal tip of the distal region;
a conductor assembly positioned in a first lead body lumen in the tubular lead body, wherein the conductor assembly comprises an ultra-high-molecular-weight polyethylene (UHMWPE) material.

13. The implantable lead of claim 12, wherein the conductor assembly comprises:
a conductor; and
an insulator surrounding the conductor.

14. The implantable lead of claim 13, wherein the insulator comprises the UHMWPE material.

15. The implantable lead of claim 14, wherein the insulator consists essentially of the UHMWPE material.

16. The implantable lead of claim 15, wherein the insulator consists of the UHMWPE material.

17. The implantable lead of claim 12, wherein the lead body comprises a polyurethane (PU) material or a silicone material.

18. A method of manufacturing an implantable lead for use with an implantable medical device (IMD), the method comprising:
forming a conductor assembly, wherein the conductor assembly comprises an ultra-high-molecular-weight polyethylene (UHMWPE) material; and
stringing the conductor assembly into a tubular lead body of the implantable lead.

19. The method of claim 18, wherein forming the conductor assembly comprises coating a conductor with the UHMWPE material.

20. The method of claim 19, further comprising shaping the UHMWPE material after coating such that the UHMWPE material has a surface texture that is configured to reduce friction with a lumen of a body of the implantable lead.
